(19) United States
(12) Patent Application Publication
(10) Pub. No.: US 2004/0170701 A1
(43) Pub. Date: Sep. 2, 2004

(54) Pharmaceutical composition comprising copper, salicylic acid, vitamin c and zinc for use in treatment of different diseases such as bacterial or viral infection

(76) Inventor: John Carter, London (GB)

Correspondence Address: BACON & THOMAS, PLLC, 625 SLATERS LANE, FOURTH FLOOR, ALEXANDRIA, VA, 22314

(21) Appl. No.: 10/472,190

(22) PCT Filed: Apr. 4, 2002
(86) PCT No.: PCT/GB02/01619

(30) Foreign Application Priority Data
Apr. 4, 2001 (GB) 0108470.6

(51) Int. Cl. A61K 33/34; A61K 33/32; A61K 33/04; A61K 33/26; A61K 31/198; A61K 31/375; A61K 31/60; A61K 31/4172

(52) U.S. Cl. 424/638; 424/639; 424/642; 424/646; 424/709; 514/159; 514/400; 514/419; 514/423; 424/702; 514/474; 514/561

(57) ABSTRACT

Therapeutic composition comprising (a) a physiologically acceptable source of assimilable copper; (b) salicylic acid or a physiologically acceptable derivative thereof; (c) vitamin C; and (d) a physiologically acceptable source of assimilable zinc; and method for the prevention or treatment of a respiratory disease, septicemia, cellulitis, AIDS, an autoimmune disease, or a bacteria or viral infection, in particular common cold or influenza, or for enhancing the immune system.

This invention relates to pharmaceutical compositions for use in the enhancement of the immune system.

Pharmaceutical compositions comprising particular copper salicylates have been proposed for use in the treatment of cancer. For example, WO 84/04922 describes the use of Cu (II) (DIPS)₂ in inhibiting cancer cell growth in vivo. DIPS is identified in WO 84/04922 as 3,5-diisopropyl salicylate. WO 95/00130 describes the use of lutein and other hydrophilic carotenoids, optionally together with one or more antioxidant or inflammatory agents including copper and salicylic acid, in particular for the treatment of coronary heart disease. WO 97/11666 describes the use of Cu(DIPS)₂ and 2-MEA in treating AIDS, cancer, auto-immune disease and microbiological infections.

It has now been unexpectedly discovered that a combination of a physiologically acceptable source of assimilable copper, salicylic acid or a derivative thereof, vitamin C and a physiologically acceptable source of assimilable zinc is particularly effective in enhancing the mammalian immune system. It is therefore useful in the treatment of diseases such as bacterial or viral infections, in particular influenza and common cold, respiratory diseases, septicaemin, cellulitis, AIDS and autoimmune disease.

The present invention therefore provides the use of:

(a) a physiologically acceptable source of assimilable copper;

(b) salicylic acid or a physiologically acceptable derivative thereof;

(c) vitamin C; and

(d) a physiologically acceptable source of assimilable zinc, in the manufacture of a medicament for use in enhancing the mammalian immune system.

Also provided is a method of treating a mammal by enhancing the immune system thereof, which method comprises administering to the mammal an effective amount of a medicament as defined above.

Also provided is a product containing components (a) to (d) as defined above for simultaneous, separate or sequential use in the enhancement of the mammalian immune system.

The present invention also provides the use of components (a) to (d) as defined above in the manufacture of a medicament for use in the prevention or treatment of a bacterial or viral infection, a respiratory disease, septicaemia, cellulitis AIDS or an autoimmune disease.

It further provides a method of treating a mammal suffering from or susceptible to a bacterial or viral infection, a respiratory disease, septicaemia, cellulitis, AIDS or an autoimmune disease, which method comprises administering to the mammal an effective amount of a medicament as defined above.

The medicament is typically for use in the prevention or treatment of, and the mammal is typically suffering from or susceptible to a bacterial or viral infection, for example common cold or influenza.

Further, the present invention provides a product containing components (a) to (d) as defined above for simultaneous, separate or sequential use in the prevention or treatment of a bacterial or viral infection, a respiratory disease, septicaemia, cellulitis, AIDS or an autoimmune disease.

Preferred medicaments of the invention comprise, in addition to components (a) to (d) listed above, one or more further components selected from:

(e) a physiologically acceptable source of assimilable manganese;

(f) a physiologically acceptable source of assimilable iron;

(g) a physiologically acceptable source of assimilable sulfur,

(h) at least one amino acid or physiologically acceptable salt thereof; and

(i) a physiologically acceptable source of assimilable selenium.

The amino acid may be any amino acid for example glutamine, glycine, cysteine, proline, serine, lysine, histidine, alanine, methionine or leucine. It can have either D- or L-stereochemistry, but is preferably an L-amino acid. Preferred amino acids include glutamine, glycine and cysteine (for example L-cysteine). Typically none, one, two or three, preferably three, of glutanine, glycine and cysteine are present in the medicament.

The physiologically acceptable salts of amino acids include salts formed with the amine group and/or with the acid group of the amino acid. Examples of suitable salts include physiologically acceptable metal salts, for example salts with copper, zinc, iron, manganese, alkali metals including sodium and potassium and alkaline earth metals including magnesium and calcium. The amino acid is typically present in its free form.

It is particularly preferred that the medicaments of the invention comprise (a), (b), (c) and (d), as defined above, and one or more, preferably two and most preferably three of components (e), (f) and (g) as defined above. Further preferred medicaments comprise components (a), (b), (c), (d), (e), (f) and (g) together with (h) and (i), as defined above.

The most preferred medicaments of the invention comprise components (a), (b), (c), (d), (e), (f), (g) and (i) and glutamine, glycine and cysteine.

Typically, the medicaments of the invention contain less than 0.35%, preferably less than 0.1% by weight carotenoid, based on the total weight of the composition. Most preferably, the medicaments of the invention contain substantially no carotenoid.

The sources of assimilable copper, manganese, iron and zinc used in the medicaments of the present invention preferably contain the metals in ionic form, e.g. metal salts with organic or inorganic acids. However, other physiologically acceptable metal compounds, e.g. metal oxides, can also be used.

Thus, a physiologically acceptable source of assimilable copper is typically a copper oxide or a salt of copper with an organic or inorganic acid. A physiologically acceptable source of assimilable manganese is typically a manganese oxide or a salt of manganese with an organic or inorganic acid. A physiologically acceptable source of assimilable iron is typically an iron oxide or a salt of iron with an organic or inorganic acid. A physiologically acceptable source of assimilable zinc is typically a zinc oxide or a salt of zinc with an organic or inorganic acid.

Suitable physiologically acceptable salts of the above metals with organic acids include salts with orotic acid, aspartic acid, gluconic acid, tartaric acid, citric acid, lactic acid, acetic acid, fumaric acid, maleic acid, malic acid, ascorbic acid, succinic acid, benzoic acid, methanesulphonic acid, ethanesulphonic acid, bcnzenesulphonic acid, p-toluenesulphonic acid and amino acids, for example glycine, glutamine or cysteine. Suitable physiologically acceptable salts of the above metals with inorganic acids include salts with hydrochloric acid, hydrobromic acid, hydriodic acid, phosphoric acid, diphosphoric acid, nitric acid or sulfuric acid, preferably hydrochloric, hydrobromic, hydroiodic, phosphoric or sulfuric acid. Such salts are available commercially or may be prepared if desired by known methods.

Preferred physiologically acceptable salts are salts with organic acids, more preferably salts with orotic acid, aspartic acid, gluconic acid, tartaric acid, citric acid, lactic acid or acetic acid and most preferred are salts with orotic or gluconic acid.

It is also preferred that the physiologically acceptable salts are water soluble, for example salts with gluconic acid.

It is particularly preferred that the physiologically acceptable salt of copper is copper orotate or copper gluconate, most preferably copper gluconate. It is particularly preferred that the physiologically acceptable salt of manganese is manganese orotate or manganese gluconate, most preferably manganese gluconate. It is particularly preferred that the physiologically acceptable salt of iron is iron orotate or iron gluconate, most preferably iron gluconate. It is particularly preferred that the physiologically acceptable salt of zinc is zinc orotate or zinc gluconate, most preferably zinc gluconate.

The sources of assimilable metals present in the medicaments of the invention may be salts of the metals with the same or different anions. Preferably, the metals are each present in the form of salts with the same anion. This anion is typically orotate or gluconate, preferably gluconate.

Salicylic acid may be present in its free acid form or as a physiologically acceptable derivative. The physiologically acceptable derivatives of salicyclic acid which may be used include compounds in which the carboxyl and/or hydroxyl function of salicylic acid has been converted into a derivative.

A physiologically acceptable derivative of salicyclic acid is typically a salicylic acid metal salt, ester or amide. Examples of suitable metal salts include alkali metal salts, for example sodium and potassium salts, and alkaline earth metal salts, for example calcium and magnesium salts. Sodium salicylate is most preferable.

Examples of suitable esters include C₁₋₆ alkyl esters, for example methyl, ethyl, propyl, butyl, pentyl or hexyl esters and particularly preferred are the methyl and ethyl esters. Examples of suitable amides are amides obtainable by reacting salicylic acid with an amine HNR₁R₂, wherein R₁ and R₂ may be the same or different and are selected from hydrogen and C₁₋₆ alkyl groups such as methyl, ethyl, propyl butyl, pentyl or hexyl. R₁ and R₂ are preferably selected from hydrogen, methyl and ethyl and most preferably both R₁ and R₂ are hydrogen.

Derivatives in which both the hydroxyl function and the caroxyl function of salicylic acid have been converted into a derivative can also be used

When the hydroxyl function of salicyclic acid is converted to a derivative it is typically converted to an ester, for example a C₁-C₆ alkyl ester such as acetyl-salicylic acid (aspirin). The medicaments of the invention may, however, contain substantially no acetyl salicylic acid (aspirin).

A particularly preferred derivative of salicylic acid is sodium salicylate.

Salicylic acid itself and suitable derivatives of it are commercially available.

Components (a) and (b) may be present as a copper salicylate complex. As used herein, a copper salicylate complex is a complex of copper and salicylic acid or a complex of copper and a said physiologically acceptable derivative of salicylic acid. Such a complex of copper and a physiologically acceptable derivative of salicylic acid may be copper (II) di-isopropyl salicylate. A suitable daily dosage of the medicament of the invention such as 4 ml of the medicament may contain less than 30 mg, preferably less than 10 mg copper (II) di-isopropyl salicylate. The medicament may contain substantially no copper (II) di-isopropyl salicylate.

Vitamin C may be present as ascorbic acid or as a salt of ascorbic acid with a metal, for example copper, manganese, zinc, iron, an alkali metal (e.g. sodium or potassium) or an alkaline earth metal (e.g. magnesium or calcium). Typically, vitamin C is present in its free acid form. Either the L- or the D form of ascorbic acid or a salt thereof may be used. Typically, the L-form is used.

Typically, sulfur is present in is elemental form and any allotropic form of sulfur may be used. Preferably, sulfur is present in the foam of sublimed sulfur or precipitated sulfur, most preferably sublimed sulfur.

Typically, selenium is present in its elemental form, as an oxide (generally SeO₂), as a selenide salt with a metal or in the form of selenium yeast. The selenide salts with a metal include alkali metal selenides and alkaline earth metal selenides, for example sodium selenide, potassium selenide, magnesium selenide and calcium selenide, in particular sodium selenide. The preferred physiologically acceptable sources of assimilable selenium are selenium yeast and sodium selenide.

The medicaments of the invention typically comprise 5 to 60, preferably 15 to 30, most preferably 20 parts by weight copper gluconate, or equivalent amount of active ingredient when a physiologically acceptable source of assimilable copper other than copper gluconate is used.

Typically, the medicaments of the invention containing salicylic acid or a physiologically acceptable derivative thereof comprise from 300 to 600, preferably 300 to 400, most preferably 350, parts by weight sodium salicylate, or equivalent amount of active ingredient when salicylic acid or a physiologically acceptable derivative thereof other than sodium salicylate is used.

Typically, the medicaments of the invention containing vitamin C comprise from 200 to 1000, preferably 300 to 500, most preferably 400, parts by weight vitamin C. Vitamin C is typically present in the medicaments of the invention in an amount significantly larger than that which is regarded as the normal minimum daily requirement for an adult.

Typically, the medicaments of the invention containing a physiologically acceptable source of assimilable zinc comprise from 5 to 60, preferably 15 to 30, more preferably 20 parts by weight zinc gluconate, or equivalent amount of active ingredient when a physiologically acceptable source of assimilable zinc other than zinc gluconate is used.

Typically, the medicaments of the invention containing a physiologically acceptable source of assimilable manganese, comprise from 5 to 60, preferably 15 to 40, more preferably 20 parts by weight manganese gluconate, or equivalent amount of active ingredient when a physiologically acceptable source of assimilable manganese other than manganese gluconate is used.

Typically, the medicaments of the invention containing a physiologically acceptable source of assimilable iron, comprise from 5 to 60, preferably 15 to 30, more preferably 20 parts by weight iron gluconate, or equivalent amount of active ingredient when a physiologically acceptable source of assimilable iron other than iron gluconate is used.

Typically, the medicaments of the invention containing a physiologically acceptable source of assimilable sulfur, comprise from 10 to 500, preferably 20 to 200, more preferably 50 to 100 parts by weight sulfur.

Typically, the medicaments of the invention containing an amino acid or a physiologically acceptable salt thereof comprise a total of from 10 to 3000, preferably from 100 to 2500, more preferably from 400 to 1800 parts by weight of amino acid(s) or salt(s) thereof. Each amino acid or amino acid salt is preferably present in an amount of from 10 to 1000, preferably 100 to 800, more preferably 400 to 600 parts by weight. The preferred medicaments of the invention comprise from 0 to 1000, preferably 100 to 800, more preferably 400 to 600 parts by weight glutamine; from 0 to 1000, preferably 100 to 800, more preferably 400 to 600 parts by weight glycine; and from 0 to 1000, preferably 100 to 800, more preferably 400 to 600 parts by weight cysteine.

Typically, the medicaments of the invention containing a physiologically acceptable source of assimilable selenium, comprise from 10 to 500, preferably 50 to 150, more preferably 80 to 120 parts by weight selenium.

The parts by weight referred to are based on the total weight of these ingredients in the medicament.

Preferably, the medicaments of the invention comprise a pharmaceutically acceptable carrier or diluent. Typically, component (a) and, if present, components (b), (c), (d), (e), (f), (g), (h) and/or (i), together with the pharmaceutically acceptable carrier(s) or diluent(s), make up at least 30% by weight of the total content of the medicament, preferably at least 50%, for example at least 60%, 70%, 80% or 90% and most preferably at least 95% by weight of the total content of the medicament.

Typically, the medicaments of the invention for the treatment or prevention of bacterial or viral infections, respiratory diseases, septicaemia, cellulitis, AIDS, autoimmune diseases and for enhancing the immune system, contain a physiologically acceptable source of assimilable copper and a physiologically acceptable source of assimilable zinc in a ratio of at least 0.15:1 (copper:zinc) by mole, preferably at least 0.2:1 and more preferably at least 0.5:1 by mole.

The medicaments of the invention may be made by first forming an intimate mixture of the sources of the assimilable metals together with the sources of sulfur and selenium and the amino acids or salts thereof, if present. This mixture, in finely ground form, can then be added to an aqueous solution or suspension of the salicylic acid or derivative thereof, if salicylic acid or a derivative thereof is used. Typically, where salicylic acid or a derivative thereof is used, from 2 to 5 ml, preferably about 3½ ml of aqueous solution or suspension is added. This solution preferably contains 5-20%, preferably about 10%, by weight of salicylic acid or derivative thereof. The vitamin C, if present, may be added before or after the salicylic acid solution, and is preferably added before the salicylic acid solution such that all of the solid ingredients are combined first.

The amounts of the active ingredients used should be calculated having regard to the intended dosage to be administered. When the medicament is to be administered orally, as is usual, a suitable dosage is about 2 ml volume for each 60 lbs of body weight of the subject to be treated. This dosage can be administered up to three times a day. The 2 ml volume dosage typically contains from 8 to 35 mg, preferably from 14 to 25 mg of copper gluconate, or an equivalent amount of active ingredient when a physiologically acceptable source of assimilable copper other than copper gluconate is used.

A suitable dosage of about 2 ml volume of the medicaments of the invention comprising salicylic acid or a physiologically acceptable derivative thereof typically contains from 90 to 1050 mg, preferably from 260 to 525 mg and most preferably about 350 mg sodium salicylate or an equivalent amount of active ingredient when salicylic acid or a physiologically acceptable derivative thereof other than sodium salicylate is used.

A suitable dosage of about 2 ml volume of the medicaments of the invention comprising vitamin C typically contains from 100 to 1200 mg, preferably from 300 to 500 mg and most preferably about 400 mg vitamin C.

A suitable dosage of about 2 ml volume of the medicaments of the invention comprising a physiologically acceptable source of assimilable zinc typically contains from 8 to 35 mg, preferably from 14 to 25 mg of zinc gluconate or an equivalent amount of active ingredient when a physiologically acceptable source of assimilable zinc other than zinc gluconate is used.

A suitable dosage of about 2 ml volume of the medicaments of the invention comprising a physiologically acceptable source of assimilable manganese, typically contains from 8 to 35 mg, preferably from 14 to 25 mg of manganese gluconate or an equivalent amount of active ingredient when a physiologically acceptable source of assimilable manganese other than manganese gluconate is used.

A suitable dosage of about 2 ml volume of the medicaments of the invention comprising a physiologically acceptable source of assimilable iron typically contains from 8 to 35 mg, preferably from 14 to 25 mg of iron gluconate or an equivalent amount of active ingredient when a physiologically acceptable source of assimilable iron other than iron gluconate is used.

A suitable dosage of about 2 ml volume of the medicaments of the invention comprising a physiologically acceptable source of assimilable sulfur typically contains from 20 to 500 mg, preferably from 50 to 200 mg of sulfur.

A suitable dosage of about 2 ml volume of the medicaments of the invention comprising one or more amino acids or physiologically acceptable salts thereof typically contains from 125 to 1500 mg, preferably from 375 to 875 mg, more preferably about 500 mg of each different amino acid or amino acid salt. Thus, a suitable dosage of about 2 ml volume of the medicaments of the invention comprising glutamine typically contains from 125 to 1500 mg, preferably from 375 to 875 mg, more preferably about 500 mg of glutamine. A suitable dosage of about 2 ml volume of the medicaments of the invention comprising glycine typically contains from 125 to 1500 mg, preferably from 375 to 875 mg, more preferably about 500 mg of glycine. A suitable dosage of about 2 ml volume of the medicaments of the invention comprising cysteine typically contains from 125 to 1500 mg, preferably from 375 to 875 mg, more preferably about 500 mg of cysteine. The total amount of amino acid or salt thereof present in a suitable dosage of about 2 ml volume is typically from 125 to 4500 mg, preferably from 375 to 2625 mg.

A suitable dosage of about 2 ml volume of the medicaments of the invention comprising a physiologically acceptable source of assimilable selenium typically contains from 25 to 300 mg, preferably from 75 to 150 mg, more preferably about 100 mg of selenium.

These figures are approximate and considerable variation in the proportions of the active ingredients is possible without losing the valuable properties of the compositions.

The medicaments of the invention may be used in human and veterinary medicine, for example in the treatment of cats and dogs. They have been found to enhance the immune system and therefore to be useful as an immunostimulant in the treatment or prevention of diseases or disorders including bacterial or viral infections, in particular common cold or influenza, respiratory disease, septicaemia, cellulitis, AIDS and autoimmune disease and other diseases and disorders which may suitably be treated or prevented by the regulation of the immune system.

The respiratory diseases which may be treated include bronchitis, bronchiectasis, asthma, bacterial or viral infections of the respiratory system, for example viral infections of the bronchi and bronchioles, pneumonia, pleurisy, respiratory distress syndrome, laryngitis and whooping cough. The autoimmune diseases which may be treated are those treatable with an immunostimulant.

Typically, a human or animal is treated by initially administering said dosage of 2 ml of the medicament of the invention, comprising active ingredients in the amounts set out above, in the form of an aqueous solution or suspension, per 60 lbs body weight of subject followed by a further dose, or optionally a half dose, of a similar solution or suspension 1 to 2 hours later. Four hours later a further dose or half dose may be given. Subsequent treatment may consist of the oral administration of a 2 ml dose or optionally a half dose (1 ml) of the said solution or suspension per 60 lbs body weight of subject once, twice or three times a day. This may be given for up to three weeks, or as required. If symptoms persist beyond three weeks, the dose may, for example, be reduced to 2 ml per 60 lbs body weight on alternate days for a further three weeks.

The active ingredients may be mixed prior to administration, or they may be administered at separate times. Typically, each of the active ingredients is administered within a period of 1 hour, preferably ½ hour, more preferably 10 minutes.

The medicaments of the invention are normally administered orally. Preferably, therefore they are suitable for oral administration. Suitable forms for oral administration include, for example, tablets, troches, lozenges, aqueous or oily suspensions, dispersible powders or granules. Preferred forms for oral administration are tablets and capsules. However, other routes of administration may be possible provided suitable precautions are taken to make the medicaments suitable for administration in the contemplated way. For example, the medicaments of the invention may be administered parenterally, whether subcutaneously, intravenously, intramuscularly, intrastemally, transdermally or by infusion techniques, or as a suppository.

The active ingredients may be administered by the same or different routes. Typically, all active ingredients are administered orally.

The medicaments of the invention may be used in combination with other pharmaceutically acceptable active agents or in conjunction with a suitable dietary regime.

The present invention also relates to compositions per se. These compositions comprise:

(a) a physiologically acceptable source of assimilable copper,

(b) salicylic acid or a physiologically acceptable derivative thereof;

(c) vitamin C;

(d) a physiologically acceptable source of assimilable zinc; and

(h) at least one amino acid or physiologically acceptable salt thereof.

In these compositions, components (a), (b), (c), (d) and (h) are as defined above. Preferred amino acids or salts thereof for use in these compositions include glutamine, glycine and cysteine, for example L-cysteine, particularly preferably glycine and cysteine. The preferred compositions comprise two or three, preferably three, amino acids. The most preferred compositions comprise two or three of glutamine, glycine and cysteine.

Typically, the compositions of the invention comprise one or more additional components selected from:

(e) a physiologically acceptable source of assimilable manganese;

(f) a physiologically acceptable source of assimilable iron;

(g) a physiologically acceptable source of assimilable sulfur; and

(i) a physiologically acceptable source of assimilable selenium.

Components (e), (f), (g) and (i) are as defined above. Preferably two, most preferably three of components (e), (f) and (g) above are present in the composition. More preferably, all of components (e), (f), (g) and (i) are present.

The most preferred compositions contain components (a), (b), (c), (d), (e), (f), (g) and (i) and glutamine, glycine and cysteine.

The preferred compounds representing components (a) to (i), and the preferred amounts of each of these components used, is as described above.

The compositions of the invention are suitable for use in the treatment of the human or animal body by therapy.

The present invention also provides the use of:

(a) a physiologically acceptable source of assimilable copper, and

(e) a physiologically acceptable source of assimilable manganese, in the manufacture of a medicament for use in the prevention or treatment of senile dementia or Alzheimer's disease. Components (a) and (e) in these medicaments are as defined above.

It further provides a method of treating a mammal suffering from or susceptible to senile dementia or Alzheimer's disease, which method comprises administering to the mammal an effective amount of a medicament as defined above.

Further, the present invention provides a product containing components (a) and (e) as defined above for simultaneous, separate or sequential use in the prevention or treatment of senile dementia or Alzheimer's disease.

Preferred medicaments for use in the treatment of senile dementia or Alzheimer's disease include, in addition to components (a) and (e) listed above, one or more further components selected from:

(b) salicylic acid or a physiologically acceptable derivative thereof;

(c) vitamin C;

(d) a physiologically acceptable source of assimilable zinc;

(f) a physiologically acceptable source of assimilable iron;

(g) a physiologically acceptable source of assimilable sulfur,

(h) at least one amino acid or a physiologically acceptable salt thereof; and

(i) a physiologically acceptable source of assimilable selenium.

Each of components (b), (c), (d) and (f) to (i) are as defined above.

The amino acid or physiologically acceptable salt thereof may be any amino acid or physiologically acceptable salt thereof as defined above. For example, the amino acid or salt thereof may be glutamine, glycine, cysteine, proline, serine, lysine, histidine, alanine, methionine or leucine. It can have either D- or L-stereochemistry, but is preferably an L-amino acid. Preferred amino acids include glutamine, glycine and cysteine (for example L-cysteine). Typically none, one, two or three, preferably three, of glutamine, glycine and cysteine are present in the medicament.

It is particularly preferred that these medicaments of the invention comprise components (a) and (e) as defined above together with component (b) and/or component (c) as defined above. Preferably all of components (a), (b), (c) and (e) are present. It is further preferred that these medicaments comprise component (f) and/or component (g) as defined above. Preferably, all of components (a), (b), (c), (e), (f) and (g) as defined above, are present. Further preferred medicaments comprise components (a), (b), (c), (d), (e), (f) and (g) together with (h) and (i), as defined above.

The most preferred medicaments of the invention for use in the treatment or prevention of senile dementia or Alzheimer's disease comprise components (a), (b), (c), (d), (e), (f), (g) and (i) and glutamine, glycine and cysteine.

The preferred compounds representing components (a) to (i), and the preferred amounts of each of these components used, is as described above. Suitable dosage regimes, suitable processes for producing these medicaments and suitable administration techniques are also as described above.

The following Examples illustrate the invention.

### EXAMPLE 1

Copper (II) gluconate (20 mg) and zinc (II) gluconate (20 mg), in finely divided form were mixed dry. Sodium salicylate solution (3.5 ml of a 10% aqueous solution) was then added followed by vitamin C (400 mg). The resulting composition is suitable for immediate oral administration.

### EXAMPLE 2

Copper (II) gluconate (20 mg), zinc (II) gluconate (20 mg) and manganese (II) gluconate (20 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 3

Copper (II) gluconate (20 mg), zinc (II) gluconate (20 mg), manganese (II) gluconate (20 mg), iron (II) gluconate (20 mg) and sublimed sulfur (50 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 4

Copper (II) gluconate (20 mg), zinc (II) gluconate (20 mg), manganese (II) gluconate (20 mg), iron (II) gluconate (20 mg), glutamine (500 mg), glycine (500 mg), cysteine (500 mg) and selenium yeast (100 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 5

Copper (II) gluconate (20 mg), zinc (II) gluconate (20 mg), manganese (II) gluconate (20 mg) and iron (II) gluconate (20 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 6

Copper (II) orotate (20 mg), zine (I) orotate (20 mg), manganese (II) orotate (20 mg), iron (11) orotate (20 mg) and sublimed sulfur (50 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 7

Copper (II) orotate (20 mg), zinc (II) orotate (20 mg), manganese (II) orotate (20 mg), iron (II) orotate (20 mg), sublimed sulfur (50 mg), glutamine (500 mg), glycine (500 mg), cysteine (500 mg) and selenium yeast (100 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 8

A human male was treated with the composition described in Example 5. He was suffering from bronchitis and showing initial symptoms of pleurisy.

He was given 4 cc of the composition (administered orally in fruit juice) followed by a further 4 cc of the composition (administered orally in fruit juice) ten minutes later. This dosage was repeated on the following day and again two days later.

A significant improvement in symptoms was noted after the first treatment and no symptoms remained after three days of treatment.

### EXAMPLE 9

A human female patient was treated using the composition described in Example 7. She was suffering from chronic severe pharyngitis and ulceration of the tongue. Symptoms included lack of energy and chronic tiredness.

The patient was given 3 cc of the composition (administered orally in fruit juice) and a further 3 cc of the composition (administered orally in fruit juice) after 20 minutes. The same dosage regime was repeated for the next two days.

A gradual regression of symptoms was noted over the first two days of treatment and a complete recovery was reported after the third day of treatment.

### EXAMPLE 10

A female human patient was treated with the composition of Example 4. She was suffering from severe swelling and inflammation of the left ankle cause by an infected insect sting.

The patient was treated with 3 cc of the composition (administered orally in apple juice). This dosage was repeated 20 minutes later and again for a third time three hours later. Two further 3 cc dosages of the composition (administered orally in apple juice) were given on the following day, the second dosage being administered an hour after the first dosage. On the third day of treatment a single 3 cc dosage of the composition was administered orally in apple juice.

An improvement in symptoms was noted three hours after the first two dosages had been administered. No further symptoms were noticeable after the third day of treatment

### EXAMPLE 11

A human female patient was treated with the composition of Example 3. She was suffering from influenza, pharyngitis and a respiratory infection.

The patient was given 4 cc of the composition (administered orally in fruit juice). A further 4 cc of the composition was administered in fruit juice after 20 minutes. After this time, a regression in symptoms was noted. The same dosage regime was repeated for the next two days, after which time all symptoms disappeared.

### EXAMPLE 12

A human male patient was treated with the composition of Example 5. He was suffering from a severe case of influenza.

4 cc of the composition was administered orally in fruit juice, followed by a second dose after 15 minutes and a further dose after one hour. A distinct improvement in the patient's condition was noted after the first day of treatment. On the following day, a further three cc doses of the composition were administered in the same manner as for the first day of treatment. All symptoms had disappeared after two days of treatment

### EXAMPLE 13

A human male patient was treated with the composition of Example 6. He was suffering from influenza.

4 cc of the composition, dissolved in fruit juice, was administered orally, followed by a further identical dose 30 minutes later. An improvement in the patient's symptoms was noted within an hour of taking the first dosage. A further two dosages of 4 cc of the composition dissolved in fruit juice were administered on the second day of treatment, again with a 30 minute break between dosages. All symptoms had disappeared after the second day of treatment

### EXAMPLE 14

A human female patient was administered with the composition of Example 6. She was suffering from influenza 4 cc of the composition, dissolved in fruit juice, was administered orally, followed by a further identical dose 30 minutes later. An improvement in the patients symptoms was noted within an hour of taking the first dosage. A further two dosages of 4 cc of the composition dissolved in fruit juice were administered on the second day of treatment, again with a 30 minute break between dosages. AU symptoms had disappeared after the second day of treatment.

### EXAMPLE 15

Copper (II) gluconate (20 mg), manganese (II) gluconate (20 mg), iron (II) gluconate (20 mg) and sublimed sulfur (50 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting composition is suitable for immediate oral administration.

### EXAMPLE 16

A human female patient was administered with the composition of Example 15. She showed symptoms of senile dementia and initial symptoms of Alzheiiner's disease. Symptoms included increasing forgetfulness and misunderstanding.

3.5 cc of the composition, dissolved in fruit juice, was administered orally on alternate days. An improvement in symptoms was observed within one week of starting treatment and further improvement was observed with continued treatment.

1. Use of:
(a) a physiologically acceptable source of assimilable copper,
(b) salicylic acid or a physiologically acceptable derivative thereof;
(c) vitamin C; and
(d) a physiologically acceptable source of assimilable zinc, in the manufacture of a medicament for use in enhancing the mammalian immune system.

2. Use of:
(a) a physiologically acceptable source of assimilable copper;
(b) salicylic acid or a physiologically acceptable derivative thereof;
(c) vitamin C; and
(d) a physiologically acceptable source of assimilable zinc, in the manufacture of a medicament for the prevention or treatment of a bacterial or viral infection, a respiratory disease, septicaemia, cellulitis, ADS or an autoimmune disease.

3. Use according to claim 3, wherein the bacterial or viral infection is common cold or influenza.

4. Use according to any one of claims 1 to 3, wherein the medicament further comprises one or more ingredients selected from:
(e) a physiologically acceptable source of assimilable manganese;
(f) a physiologically acceptable source of assimilable iron;
(g) a physiologically acceptable source of assimilable sulfur;
(h) at least one amino acid or physiologically acceptable salt thereof; and
(i) a physiologically acceptable source of assimilable selenium.

5. Use according to claim 4, wherein the at least one amino acid or physiologically acceptable salt thereof is glutamine, glycine and/or cysteine.

6. Use according to any one of claims 1 or 3, wherein the medicament further comprises:
(e) a physiologically acceptable source of assimilable manganese,
(c) a physiologically acceptable source of assimilable iron; and
(d) a physiologically acceptable source of assimilable sulfur.

7. Use according to any one of claims 1 to 3 or 6, wherein the medicament further comprises glutamine, glycine, L-cysteine and a physiologically acceptable source of assimilable selenium.

8. Use according to any one of the preceding claims wherein the said metals are present in the form of oxides or salts with organic or inorganic acids.

9. Use according to claim 8, wherein the salts are the same or different and are selected from orotates, aspartates, gluconates, tartrates, citrates, lactates, acetates, chlorides, bromides, iodides, phosphates and sulfates.

10. Use according to any one of the preceding claims wherein components (a) and (b) are present as a copper salicylate complex.

11. Use according to any one of claims 1 to 9, wherein component (b) is sodium salicylate.

12. A composition comprising:
(a) a physiologically acceptable source of assimilable copper,
(b) salicylic acid or a physiologically acceptable derivative thereof;
(c) vitamin C;
(d) a physiologically acceptable source of assimilable zinc; and
(h) at least one amino acid or physiologically acceptable salt thereof as defined in claim 4 or 5.

13. A composition according to claim 12, which further comprises one or more compounds selected from:
(e) a physiologically acceptable source of assimilable manganese;
(f) a physiologically acceptable source of assimilable iron;
(g) a physiologically acceptable source of assimilable sulfur; and
(i) a physiologically acceptable source of assimilable selenium.

14. A composition according to claim 13, which comprises components (a), (b), (c), (d), (e), (f), (g) and (i) and glutamine, glycine and cysteine.

15. A composition according to any one of claims 12 to 14 for use in the treatment of the human or animal body by therapy.

16. A product containing:
(a) a physiologically acceptable source of assimilable copper,
(b) salicylic acid or a physiologically acceptable derivative thereof as defined in any one of claims 1, 10 or 11;
(c) vitamin C; and
(d) a physiologically acceptable source of assimilable zinc; for simultaneous, separate or sequential use in the enhancement of the mammalian immune system.

17. A product containing:
(a) a physiologically acceptable source of assimilable copper,
(b) salicylic acid or a physiologically acceptable derivative thereof as defined in any one of claims 1, 10 or 11;
(c) vitamin C; and
(d) a physiologically acceptable source of assimilable zinc;
for simultaneous, separate or sequential use in the prevention or treatment of a bacterial or viral infection as defined in claim 2 or claim 3, a respiratory disease, septicaemia, cellulitis, AIDS or an autoimmune disease.

18. A product according to claim 16 or 17, which further comprises one or more ingredients selected from:
(e) a physiologically acceptable source of assimilable manganese;
(f) a physiologically acceptable source of assimilable iron;
(g) a physiologically acceptable source of assimilable sulfur;
(h) at least one amino acid or physiologically acceptable salt thereof as defined in claim 4 or claim 5; and
(i) a physiologically acceptable source of assimilable selenium.

19. A product according to claim 16 or 17, which further comprises:
(e) a physiologically acceptable source of assimilable manganese;
(d) a physiologically acceptable source of assimilable iron; and
(f) a physiologically acceptable source of assimilable sulfur.

20. A product according to any one of claims 16, 17 or 19 which further comprises glutamine, glycine, L-cysteine and a physiologically acceptable source of assimilable selenium.

21. A product according to any one of claims 16 to 20, wherein the metals are present in the form of oxides or orotate, aspartate, gluconate, tartate, citrate, lactate, acetate, chloride, bromide, iodide, phosphate or sulphate salts, the said metals being present in the same or different form(s).

22. Use of:
(a) a physiologically acceptable source of assimilable copper, and
(e) a physiologically acceptable source of assimilable manganese; in the manufacture of a medicament for use in the prevention or treatment of senile dementia or Alzheimer's disease.

23. Use according to claim 22, wherein the medicament further comprises one or more ingredients selected from:
(b) salicylic acid or a physiologically acceptable derivative thereof as defined in any one of claims 1, 10 or 11;
(c) vitamin C;
(d) a physiologically acceptable source of assimilable zinc;
(f) a physiologically acceptable source of assimilable iron;
(g) a physiologically acceptable source of assimilable sulfur,
(h) at least one amino acid or physiologically acceptable salt thereof as defined in claim 3 or claim 4; and
(i) a physiologically acceptable source of assimilable selenium.

24. Use according to claim 22, wherein the medicament further comprises:
(b) salicylic acid or a physiologically acceptable derivative thereof as defined in any one of claims 1, 10 or 11; and
(c) vitamin C.

25. Use according to claim 22 or claim 24, wherein the medicament further comprises:
(f) a physiologically acceptable source of assimilable iron; and
(g) a physiologically acceptable source of assimilable sulfur.

26. Use according to any one of claims 22, 24 or 25, wherein the medicament further comprises a physiologically acceptable source of assimilable zinc, glutamine, glycine, L-cysteine and a physiologically acceptable source of assimilable selenium.

27. Use according to any one of claims 22 to 26, wherein the metals are present in the form of oxides or orotate, aspartate, gluconate, tartrate, citrate, lactate, acetate, chloride, bronide, iodide, phosphate or sulphate salts, the said metals being present in the same or different form(s).

28. A product containing:
(a) a physiologically acceptable source of assimilable copper; and
(e) a physiologically acceptable source of assimilable manganese, for simultaneous, separate or sequential use in the prevention or treatment of senile dementia or Alzheimer's disease.

29. A product according to claim 28, which further comprises one or more ingredients selected from:
(b) salicylic acid or a physiologically acceptable derivative thereof as defined in any one of claims 1, 10 or 11;
(c) vitamin C;
(d) a physiologically acceptable source of assimilable zinc;
(f) a physiologically acceptable source of assimilable iron;
(g) a physiologically acceptable source of assimilable sulfur,
(h) at least one amino acid or physiologically acceptable salt thereof as defined in claim 4 or claim 5; and
(i) a physiologically acceptable source of assimilable selenium.

30. A product according to claim 28, which further comprises:
(b) salicylic acid or a physiologically acceptable derivative thereof as defined in any one of claims 1, 10 or 11; and
(c) vitamin C.

31. A product according to claim 28 or 30, which further comprises:
(f) a physiologically acceptable source of assimilable iron; and
(g) a physiologically acceptable source of assimilable sulfur.

32. A product according to any one of claims 28, 30 or 31, which further comprises a physiologically acceptable source of assimilable zinc, glutamine, glycine, L-cysteine and a physiologically acceptable source of assimilable selenium.

33. A product according to any one of claims 28 to 32, wherein the metals are present in the form of oxides or orotate, aspartate, gluconate, tatrate, citrate, lactate, acetate, chloride, bromide, iodide, phosphate or sulphate salts, the said metals being present in the same or different form(s).
